(19) United States
(12) Patent Application Publication
(10) Pub. No.: US 2015/0164706 A1
(43) Pub. Date: Jun. 18, 2015

(54) ANTI-IRRITANT DISPOSABLE DIAPER

(71) Applicants: Chaim BEN NATAN, (US); Amichai BEN NATAN, (US)

(72) Inventors: Chaim Ben-Natan, Kfar Chabad (IL); Amichai Ben-Natan, Tzfat (IL)

(21) Appl. No.: 14/413,010

(22) PCT Filed: Jul. 11, 2013
(86) PCT No.: PCT/IL13/50593

§ 371 Date: Jan. 6, 2015

Related U.S. Application Data
(60) Provisional application No. 61/670,608, filed on Jul. 12, 2012.

(51) Int. Cl. A61F 13/495; A61F 13/494

(52) U.S. Cl. CPC A61F 13/495; A61F 13/49413; A61F 2013/4956

(57) ABSTRACT

An improved anti-irritant disposable diaper, comprising a semi-rigid element defining and maintaining a gap extending along a user contour and a waste containment means integrally formed within a crotch area and disposed below the gap for receiving excreted waste, thus substantially preventing contact of the skin of the user with waste, so as not to cause rashes.

### CROSS-REFERENCE TO RELATED APPLICATION

The present invention claims the priority date of U.S. Provisional Patent Application No. 61/670,608 filed on 12 Jul. 2012, by the one of the co-applicants herein.

### FIELD OF THE INVENTION

The present invention relates to disposable diapers, in particular to diapers constructed for protection from skin irritation.

### BACKGROUND OF THE INVENTION

Use of disposable diapers has provided a convenient and sanitary solution for parents of infants and care-givers of the elderly. However, the plastic coating used on the outside of the absorbent layers, while helping to prevent leaks, also causes the waste to be left on the skin of the diaper user, and creates a breeding ground for rash-causing micro-organisms.

Many advances have been made in diaper technology such as the use of gels to lock in liquid waste and prevent it from irritating the infant's skin. These advances have improved the use of disposable diapers. However, many diaper-users continue to suffer from irritations and rashes. A less apparent problem are the rashes caused by contact with solid waste.

The contact between solid or semi-solid waste (hereinafter “solid waste”) waste and the skin can cause irritation and even pain, especially if the diaper-user has sensitivity to types of food. Also, care-givers suffer from discomfort and repulsion caused by changing soiled diapers and having to scrub the waste off the patient's skin with soap and water. In addition, solid waste often escapes from the diaper onto the legs and clothes.

Additionally, disposable diapers can be expensive to use and are perceived as wasteful. Dirty diapers must be changed as soon as possible so as to prevent rashes, but often the diaper is not wet and its absorbent capacities have not been realized. Because disposable diapers are not biodegradable, it is necessary to find ways of conserving the use of diapers.

A solution for the above-mentioned problems has been proposed by the applicant in U.S. Pat. No. 6,464,673, disclosing a diaper having a gap between the diaper and the skin surface, and a solid waste containment means retained in the gap. The present invention is an improvement of the above-mentioned patented invention.

### SUMMARY OF THE INVENTION

The present invention overcomes the problems associated with the prior art by providing an anti-irritant disposable diaper construction which contains solid waste and distances the surface of the absorbent material from the skin of the user.

The present invention provides a sanitary device, typically a diaper or sanitary garment or the like, that contains solid waste, so as to prevent its coming in contact with the skin of the user, so as not to cause rashes. This also makes cleaning the diaper-user easier, during the diaper changing process, since the skin remains relatively clean.

The present invention is useful for both infants and the elderly who cannot control their bowel movements. The inventive diaper allows the diaper-user to be more mobile, since the solid waste is contained inside the diaper is not allowed to escape onto the legs and clothes, as often occurs when using a regular diaper.

The contact between the waste and the skin is reduced to a minimum, if any, so that any feeling of discomfort is decreased. It is especially useful in places and situations where it is difficult to change the diaper, and the user is forced to remain with the soiled diaper for an extended period of time which by then, normally, would cause irritation to the skin.

The present invention is also especially useful for elderly patients in a nursing home, who are treated by care-givers whom also change their diapers. The process of changing diapers causes repulsion and disgust on behalf of the changer. The inventive diaper will minimize the care-giver's exposure to the solid waste during changing, because the waste will be safely contained inside a dedicated pocket.

The inventive diaper may also reduce the number of staff needed to care for the patients, since a great deal of time and work, put into cleaning the patients, can be saved.

These sanitary devices are also economical and environmentally-friendly since they may be used multiple times, thus reducing the amount of diaper waste.

In a preferred embodiment of the present invention there is provided an anti-irritant disposable diaper comprising:

a diaper element having an interior, a top area, a front area, a crotch area, a rear area, leg cuffs and closing means;
a semi-rigid element extending from a top front edge of said front area of said diaper element, along said crotch area, to a top rear edge of said rear area, said semi-rigid element defining and maintaining a gap extending along a user contour; and
a solid waste containment means disposed below the gap for receiving excreted solid waste, thus preventing contact of a user's skin with waste.

In this embodiment, the diaper has a front area which closely contacts the body and its purpose is absorbing fluid waste, and extends to a crotch area having a solid waste containment means, resembling a pocket, above which there is a gap which extends along a user contour. The crotch area extends to a rear area. The diaper is provided with a semi-rigid element which is made out of a sponge, or any other suitable material. The semi-rigid element is a longitudinal element having joints formed therein along its length so as to define three sections, each with a different orientation: the front section is substantially vertical, the middle section is substantially horizontal and the rear section is substantially vertical. The element's purpose is defining and maintaining the structure of the pocket. The semi-rigid element may be shaped in a shape other than that described above, such as a prism, for instance, as described in an alternative embodiment of the present invention. The sides of the pocket area are made out of the external layer of a regular diaper, not including the absorbing matter.

The substantially-rigid element must be soft and comfortable to the user on the one hand, and on the other hand must be sufficiently rigid in order to maintain the structure, i.e. entrance and depth, of the pocket. Another function of the element is to press the front area upwards in such a way that it closely contacts the body, so that while sitting, the solid waste will not come in contact with the body, and will not have the opportunity to spread to the front area.

In a further embodiment of the present invention, there is provided a wrapping of the substantially-rigid element with the absorbing material of the diaper. The absorbing material will transfer the liquid waste also to the rear area, and will also help in absorbing fluid fecal matter so the skin will remain clean during sitting.

In yet another embodiment of the present invention there is provided a substantially-rigid element that is attached to the inventive diaper but is inserted inside the diaper so it is not visible, or is inserted all the way through the diaper so it is external to the diaper.

In an additional embodiment of the present invention, there is provided a pocket in the crotch area, which is closed from the top by a partition with an X-shaped opening, so that the solid waste enters the pocket through the X-shaped opening.

In a further additional embodiment of the present invention, there is provided a pocket in the crotch area, where the semi-rigid element is shaped in such a way that while sitting, the front area folds over the pocket of the crotch area, so the pocket is sealed and its contents cannot contact the skin. When the diaper-user stands up again the pocket opens up once again.

In another embodiment of the present invention, there are provided cuffs having a sheet of material extending into the crotch area, internally proximal and similar to the leg cuffs, which are provided to contain the waste and substantially prevent it from contacting the skin. The cuffs are positioned diagonally to each other at various angles, or parallel to each other, at varying proximities to each other and at various heights.

In yet another embodiment of the present invention there is provided a cover for the pocket area, which can be pulled over the pocket by pulling a string external to the diaper, so that the solid waste captured in the pocket will not touch the skin. The cover may be pulled open by pulling another external string to expose the pocket in the case of re-use by the diaper user. This feature is especially suitable for the elderly, who can open and close the cover themselves, more than once if needed, taking into consideration the diaper's capacity limit.

In a further embodiment of the present invention, there is provided inner cuffs connected to a pullable string, so that when the string is pulled, the inner cuffs move towards each other and are joined thus covering said pocket.

In another embodiment of the present invention there is provided one or more scented stickers placed externally on the diaper, for use when it is not possible to change the diaper.

In yet another embodiment of the present invention, there is provided an extended semi-rigid element, forming a larger pocket in the crotch area.

In a further embodiment of the present invention, there are provided inner cuffs filled with air, so that when the diaper-user sits, the pressure on the cuffs will cause them to expand to a point where the crotch-area is sealed.

In yet another embodiment of the present invention there is provided a semi-rigid element in the crotch-area, fabricated from the same material as the outer-layer of the diaper, and is filled with air. This design may be more economical for manufacturing.

In another embodiment of the present invention, there is provided a special material for added absorbency which also leads the urine along the diaper so that the urine is dispersed evenly over the diaper. This material may be a mixture of Superabsorbent polymers (SAP) and Lignocellulosic fibrous material (Pulp), or any other suitable absorbent material.

In an additional embodiment of the present invention, there is provided a thickening of the front or rear area or both, for added absorbency.

In a further embodiment of the present invention, there is provided a stitch extending along the length of the bottom side of crotch area, and the stitch lifts the middle of the crotch area resulting in a compartment for receiving solid waste on either side of the stitch for substantially preventing contact of a user's skin with the solid waste.

In an additional embodiment of the present invention, there is provided a semi-rigid element positioned across the width of the crotch area for the purpose of maintaining the structure of the pocket.

In another embodiment of the present invention, there is provided a semi-rigid element that is shaped as a longitudinal section, placed on the rear area and on the front area, and wherein the two longitudinal sections are spaced apart from each other, for the purpose of maintaining the structure of the pocket.

In another additional embodiment of the present invention, there is provided an anti-irritant disposable diaper comprising:
a diaper element having an interior, a top area, a front area, a crotch area, a rear area, leg cuffs and closing means;
a gap extending along a user contour and above said crotch area;
a solid waste containment means integrally formed within said crotch area and disposed below said gap for receiving excreted solid waste, thus substantially preventing contact of a user's skin with solid waste; and
at least one cuff placed internally and proximate to said leg cuffs, and over said crotch area, wherein said inner cuff comprises a sheet of material for substantially covering solid waste received in said containment means, so that the solid waste is substantially prevented from contacting the user's skin.

In a further embodiment of the present invention, there is provided a sanitary device for providing protection against irritation to the skin, said device comprising:
a disposable diaper, comprising:
   a diaper element having an interior, a top area, a front area, a crotch area, a rear area, leg cuffs and closing means;
   a semi-rigid element extending from a top front edge of the front area of the diaper element, along the crotch area, to a top rear edge of the rear area, the semi-rigid element defining and maintaining a gap extending along a user contour; and
   a waste containment means disposed below the gap for receiving excreted waste, thus substantially preventing contact of a user's skin with waste.

Additional features and advantages will become apparent from the following drawings and description.

### BRIEF DESCRIPTION OF THE DRAWINGS

For a better understanding of the invention with regard to the embodiments thereof, reference is made to the accompanying drawings, in which like numerals designate corresponding elements or sections throughout and in which:

FIG. 1 shows a perspective view of the diaper of the present invention and its component parts;

FIG. 2 shows a cross sectional view of FIG. 1, showing the side of the inner cuffs (as described in FIG. 5);

FIG. 3a shows a cross-sectional view of FIG. 1, showing a cross section of the semi-rigid element;

FIG. 3b shows a cross sectional view of FIG. 1, showing a cross section of a two-piece alternative embodiment of the semi-rigid element;

FIG. 4 shows a cross-sectional side view of another preferred embodiment of the diaper of the present invention, illustrating a partition covering the crotch-area;

FIG. 5 shows a side view of an alternative embodiment of the diaper of the present invention in which the inner-cuffs can be pulled together by pulling a string on the side;

FIG. 6 shows the diaper of FIG. 5 in which the inner-cuffs are pulled together;

FIG. 7 shows the diaper of FIG. 1 illustrating a partition, covering the crotch-area, which is being pulled open from the sides;

FIG. 8 shows the diaper of FIG. 1 illustrating a partition covering the crotch-area being pulled closed from the sides;

FIG. 9 shows a cross-sectional view of an alternative embodiment of the present invention provided with a thicker front and rear padding for enhanced absorbency;

FIG. 10 shows another embodiment of FIG. 1 in which the semi-rigid element is extended so that the pocket formed in the crotch area is larger;

FIG. 11 shows a cross-sectional top view of the diaper of FIG. 10;

FIG. 12 shows an additional embodiment of the diaper of FIG. 1, with the inner cuffs filled with air;

FIG. 13 shows the diaper of FIG. 12, showing the cuffs expanded by applied pressure from the top;

FIG. 14 shows a bottom view of an additional embodiment of the diaper of the present invention, illustrating a stitch formed along the diaper creating side-pockets;

FIG. 15 shows a perspective view of another preferred embodiment of the diaper of the present invention, illustrating a prism-shaped element covering the crotch-area;

FIG. 16 shows a perspective view of another preferred embodiment of the diaper of the present invention, illustrating a portion of the diaper fabricated out of absorbent material;

FIG. 17 shows a perspective view of the diaper of FIG. 1, showing the inner cuffs parallel and close to each other; and

FIG. 18 shows a cross-sectional view of another preferred embodiment of the diaper of the present invention, showing a semi-rigid element positioned across the width of the diaper.

### DETAILED DESCRIPTION OF THE INVENTIVE EMBODIMENTS

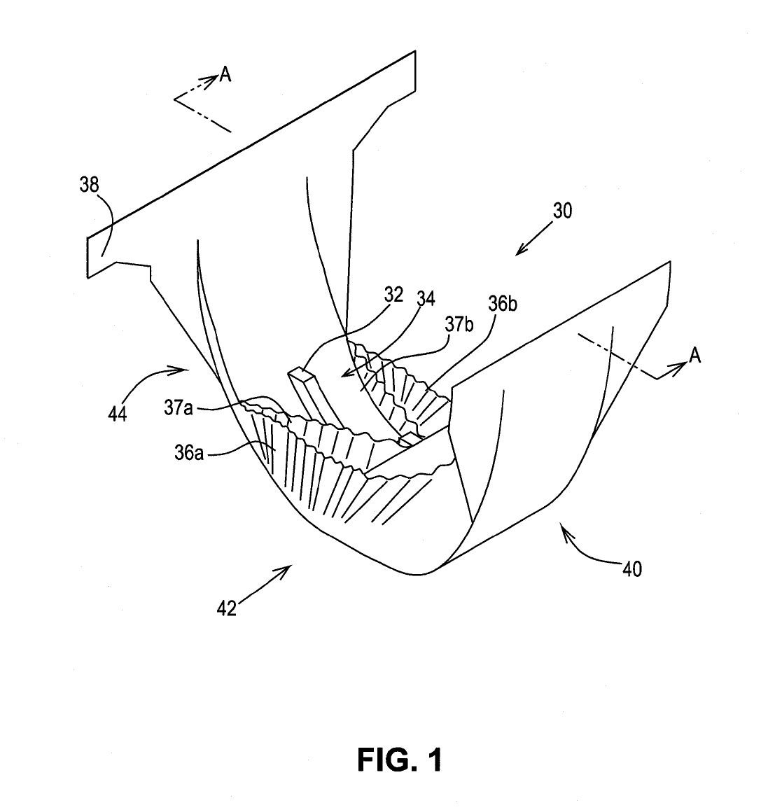

Referring now to FIG. 1, there is shown diaper 30 constructed in accordance with the principles of the present invention. Diaper 30 is constructed of a front area 40 and a rear area 44, when in the middle is crotch area 42. The rear area 44 comprises two fasteners 38, one on each side at the ends of rear area 44 ends (as in conventional diapers), for closing the diaper. Crotch area 42 comprises side cuffs 36a, 36b, one on each side (as in conventional diapers), and inner cuffs 37a, 37b, one on each side.

Placed in crotch area 42 is semi-rigid element 32 which is shaped as a longitudinal element having joints formed therein along its length so as to define three sections, each with a different orientation: the front section is substantially vertical, the middle section is substantially horizontal and the rear section is substantially vertical, and its purpose is defining and maintaining the depth of pocket 34 formed in crotch area 42. Semi-rigid element 32 may be shaped in a shape other than the three-sectional element described hereinabove. Semi-rigid element 32 is fabricated from a sponge, or any other suitable material rigid enough to maintain the pocket-form while being soft enough for comfort. The pocket 34 is important for creating a gap between the waste and the user's skin to avoid irritation.

The sizes of the diapers are adapted to fit various sizes of people, from new-born babies to the elderly, and so the dimensions of the semi-rigid element 32 are varied to fit these different-sized diapers, in order to form a suitable pocket 34 in the crotch area 42.

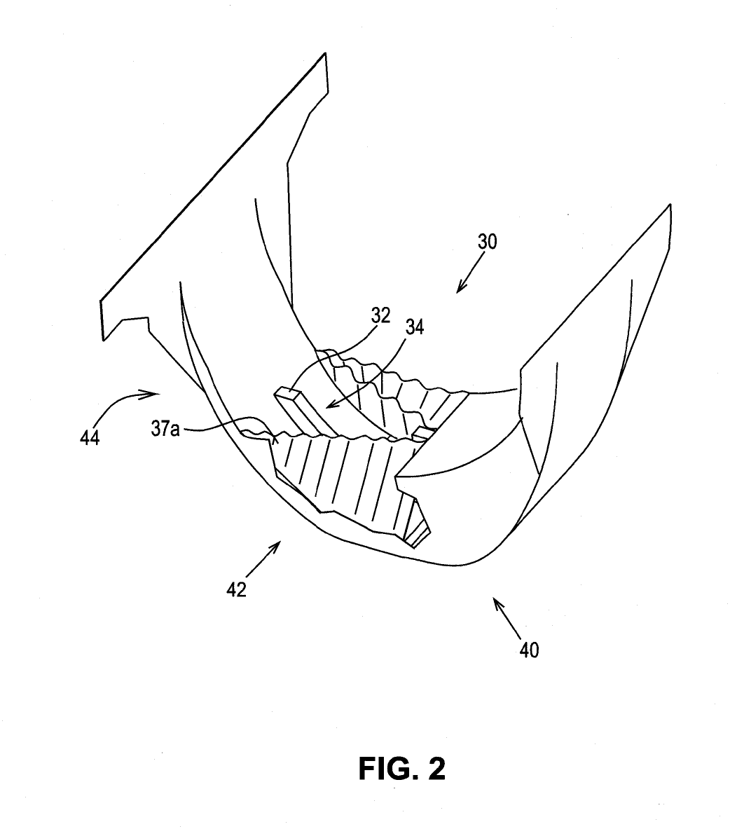

Referring now to FIG. 2 there is shown a cross-sectional view of diaper 30 of FIG. 1, taken along section lines A-A illustrating the side of inner cuff 37a. Inner cuffs 37a-b are placed diagonally from the middle of the diaper 30 to the rear end 44 in a widening fashion, and the edges of inner cuffs 37a-b are slightly slanted inwards, so that when the user sits down, the cuffs 37a-b will be pressed downwards and will not allow the waste to contact the body. Inner cuffs 37a-b are in close contact with the body, and are made sufficiently long so that the waste is securely contained inside the diaper 30. Inner cuffs 37a-b can be in close contact with the body, or not in close contact.

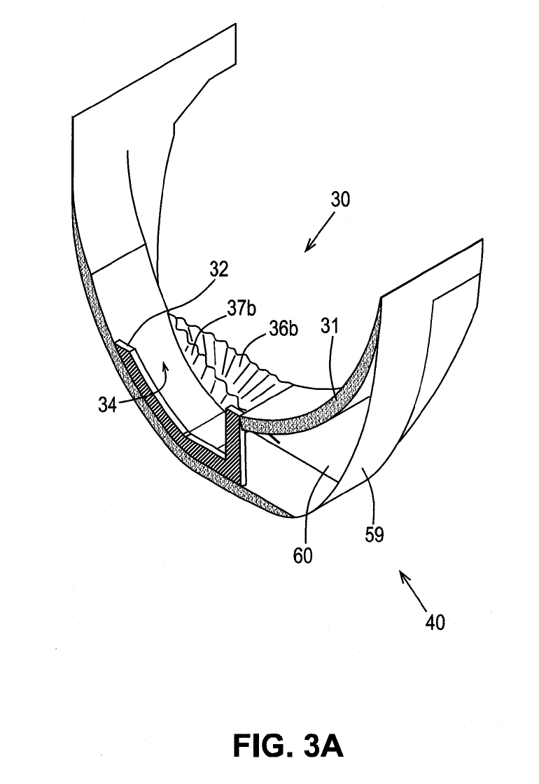

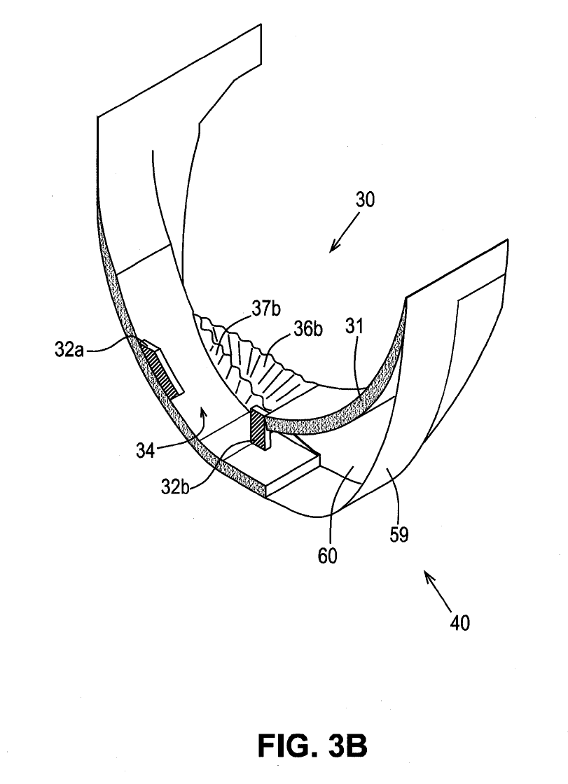

Referring now to FIG. 3A there is shown a cross-section of the semi-rigid element 32 of diaper 30. In front area 40 there is shown part 31 which contacts the user's body closely for absorbing liquid waste. In between part 31 and the outer layer 59 of diaper 30, there is formed a gap 60, which is able to contain solid waste, for the possibility when a large amount of waste is excreted. By having gap 60 formed in diaper 30, solid waste will not leak out of the diaper, but will advance naturally to gap 60.

Outer layer 59 may be padded with absorbing material for enhanced absorbency (not shown).

Referring now to FIG. 3B there is shown a cross-section of diaper 30 showing a cross section of semi-rigid element 32a positioned in crotch area 42 near rear area 44, and semi-rigid element 32b positioned in crotch area 42 near front area 40. Both elements 32a and 32b substantially maintain the structure of pocket 34, without using the middle section of semi-rigid element 32, thereby reducing manufacturing cost.

In the following Figs. below, additional embodiments of diaper 30 are shown, with each alternative embodiment labeled as 30a, 30b, 30c, etc.

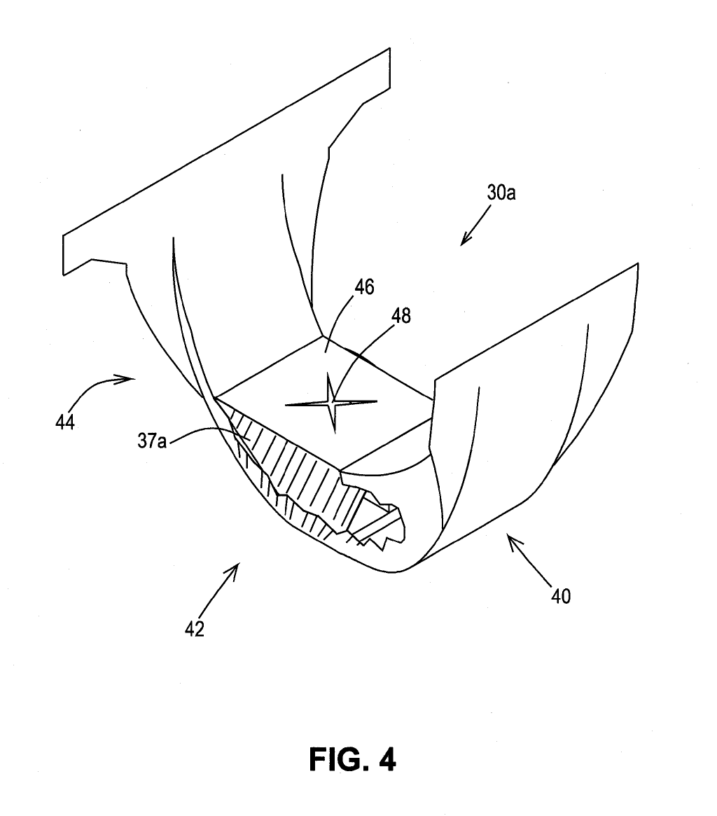

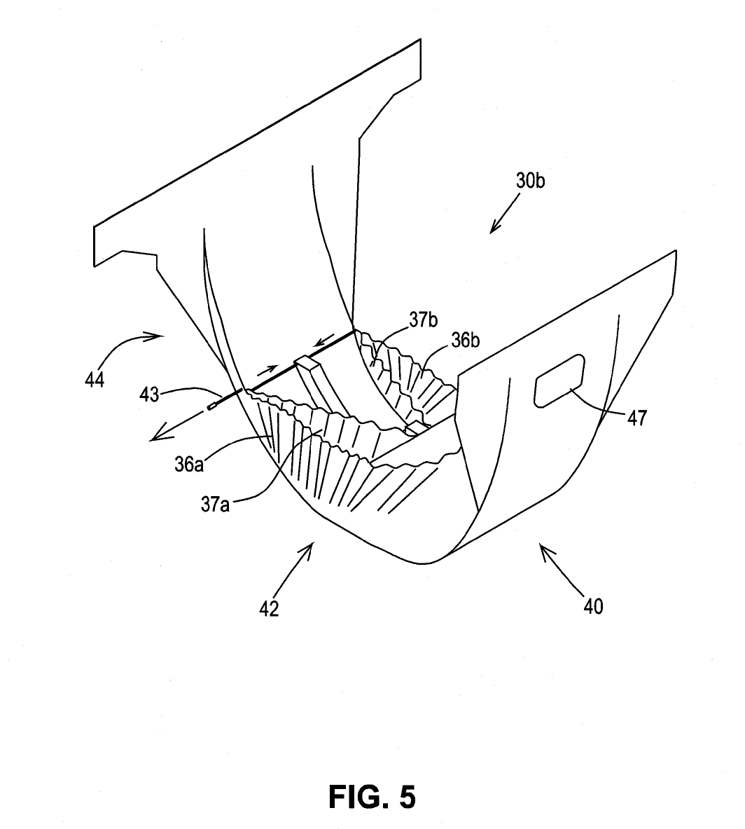

Referring now to FIG. 4 there is shown diaper 30a, having a partition 46 covering pocket 34, with an opening 48 for solid waste to pass through into pocket 34.

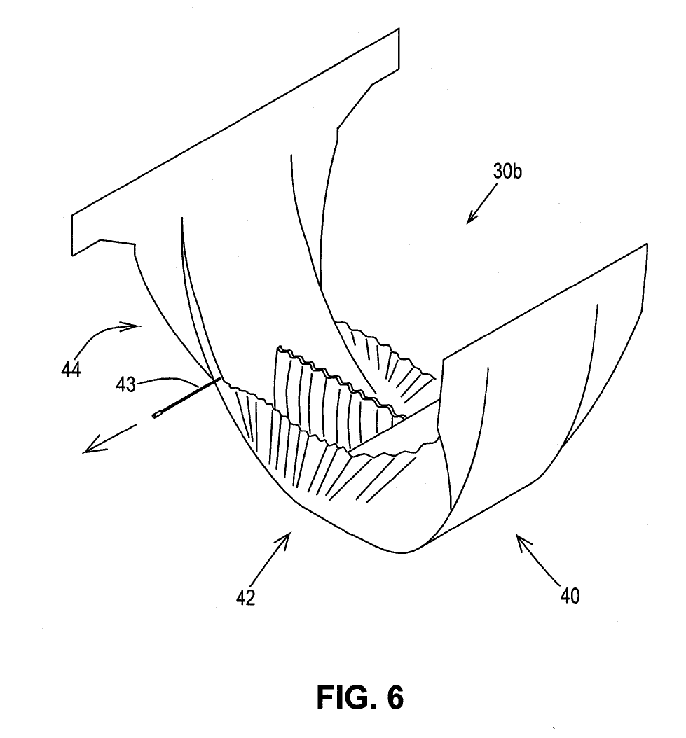

Referring now to FIG. 5 there is shown diaper 30b with inner cuffs 37a, 37b attached to string 43. When string 43 is pulled, inner cuffs 37a, 37b are pulled together so pocket 34 is closed and the waste is contained inside it.

A scented sticker 47 may be placed outside the diaper, in any place, for instance the front area 40. It can be used on occasions when the diaper cannot be changed for the time being, and then the sticker 46 may be peeled of its seal and a pleasant scent is released.

Referring now to FIG. 6 there is shown diaper 30b with inner cuffs 37a, 37b closed by pulling string 43.

The inner cuffs 37a-b are not limited to one embodiment, but can be added to any embodiment of the present invention.

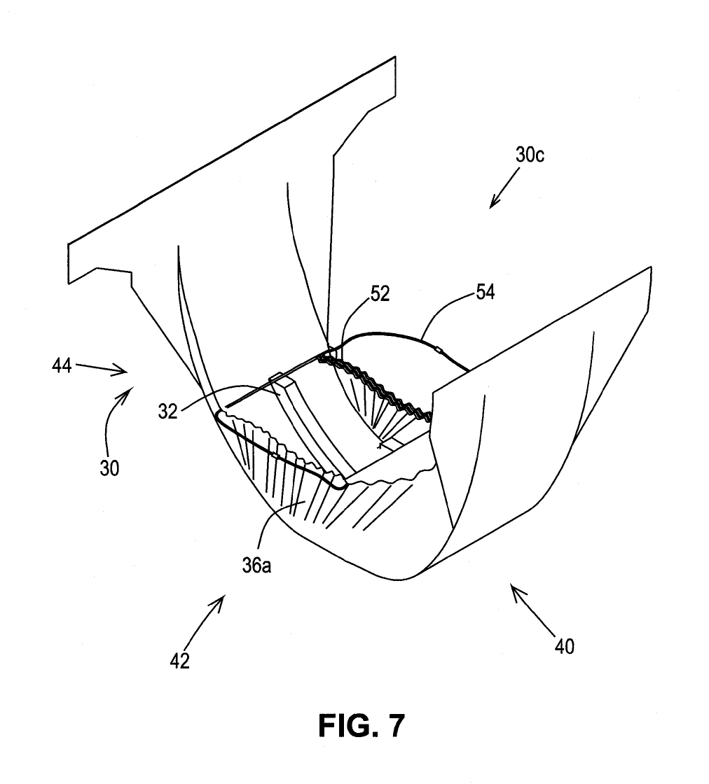

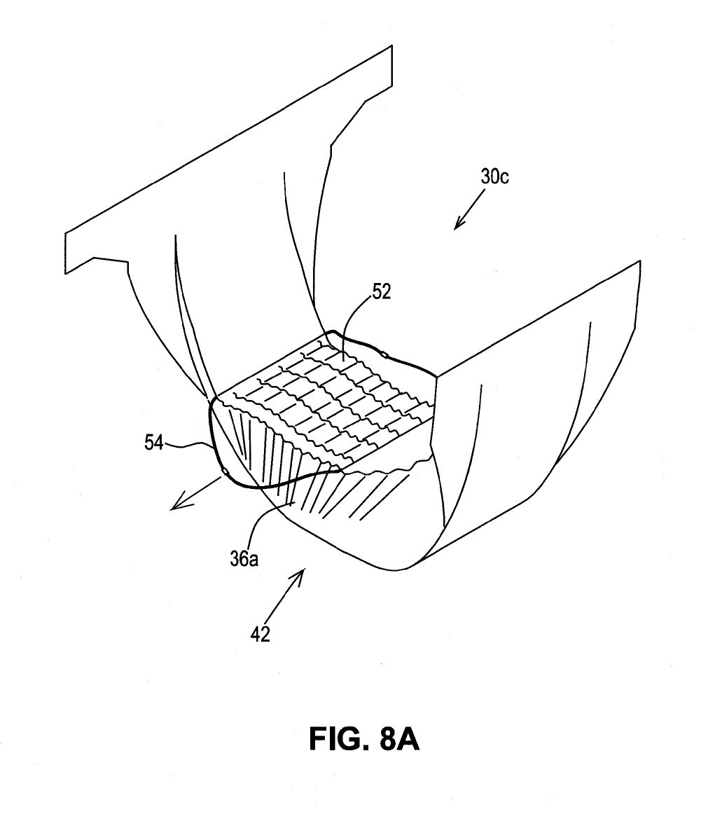

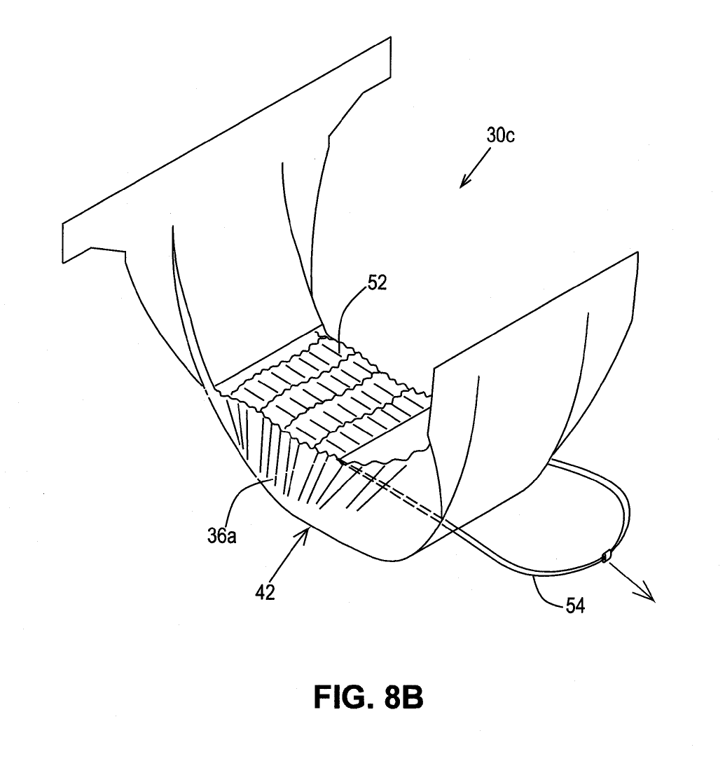

Referring now to FIG. 7, there is shown diaper 30c with a slidable partition 52 attached to string 54, which is external to the diaper, to be pulled over crotch area 42 (as shown in FIG. 8a).

Referring now to FIG. 8a, there is shown diaper 30c with slidable partition 52 pulled over crotch area 42.

Slidable partition 52 may be pulled over crotch area 42 by pulling string 54 from side cuff 36a, thus causing partition 52 to stretch from its tightly-bunched position to its spread-out position, extending from cuff 36b (not shown) to cuff 36a. Slidable partition 52 may be retracted to its tightly-bunched position by pulling string 54 from the opposite side, i.e., the nearest cuff 36b. Once partition 52 is retracted, crotch area 42 is revealed. This may be done in the case the user needs to re-use the diaper 30c once more.

Partition 52 is provided so that the solid waste captured in pocket 34 will not touch the skin and irritate it. This feature is especially suitable for the elderly, who can open and close the slidable partition 52 themselves, more than once if needed, taking into consideration the diaper's capacity limit.

There is an option (not shown) for the slidable partition 52 to be pulled over crotch area 42, from inner cuff 37b to inner cuff 37a. The slidable partition 52 is positioned in the most convenient place, so that the string 54 will not become soiled.

In this particular embodiment, the inner cuffs 37a-b are not necessary, and may be part of the construction or not.

Referring now to FIG. 8b, there is shown an alternative embodiment of diaper 30c, with slidable partition 52 to be pulled over crotch area 42, from the direction of the rear area 44 to front area 40.

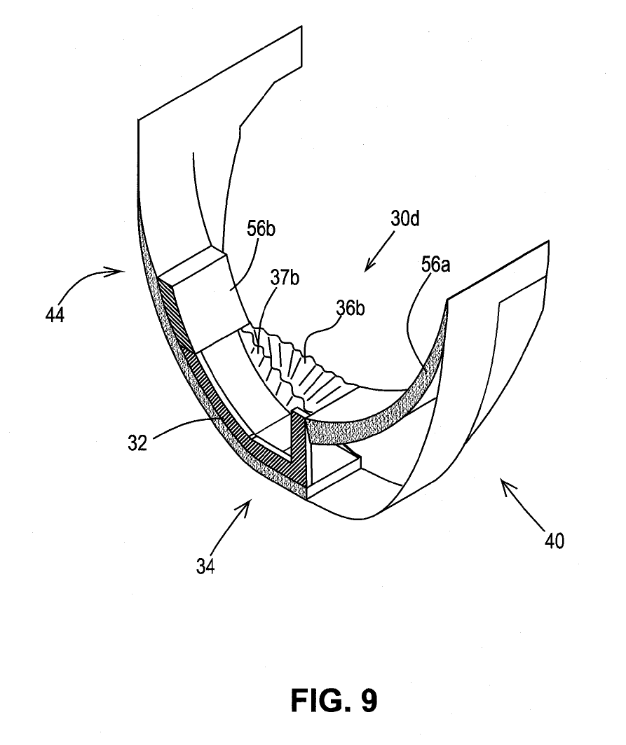

Referring now to FIG. 9, there is shown a cross-sectional view of an alternative embodiment of the present invention, wherein diaper 30d is provided with a thicker front padding 56a for enhanced absorbency of urine, especially convenient for night time. There is also provided a thicker rear padding 56b.

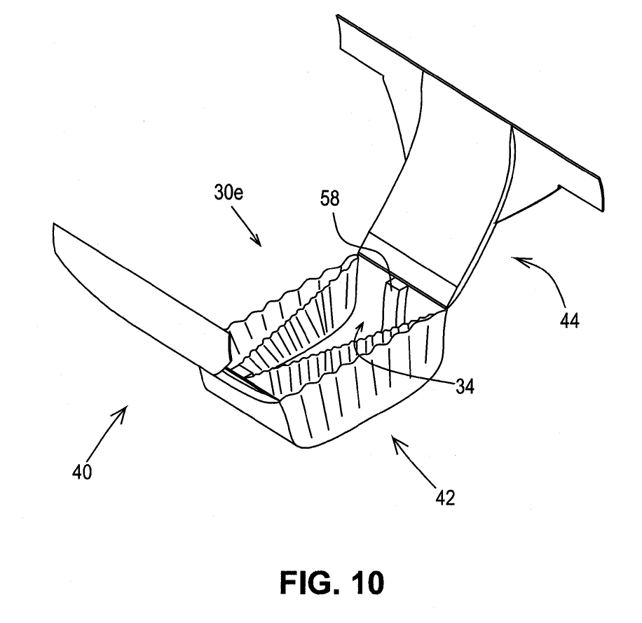

Referring now to FIG. 10, there is shown diaper 30e with an extended substantially-rigid element 58, which forms a larger pocket area 34, so that the solid waste contained there is kept farther away from the skin. The rear area 44 of diaper 30e will be slightly protruding due to the length of the extended element 58, so that the waste will easily enter pocket 34.

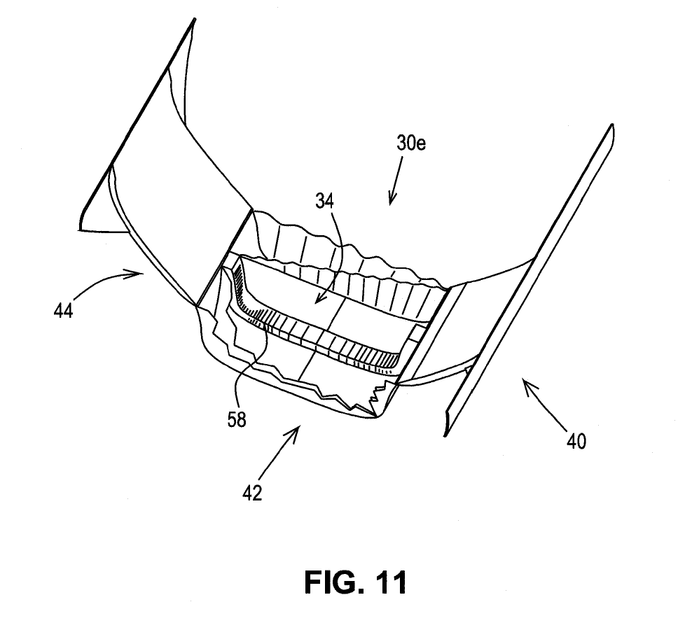

Referring now to FIG. 11, there is shown a cross-sectional top view of diaper 30e of FIG. 10, revealing the extended substantially-rigid element 58, and illustrating the large pocket-area 34 formed by element 58.

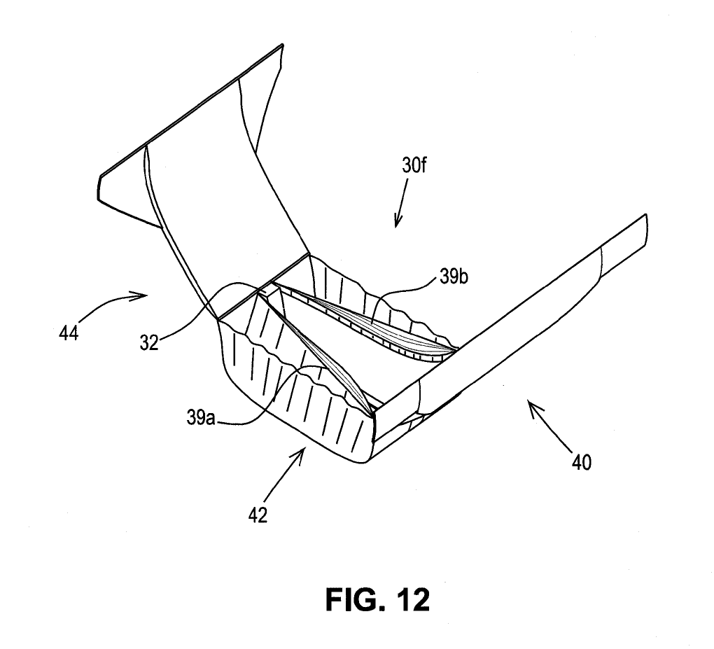

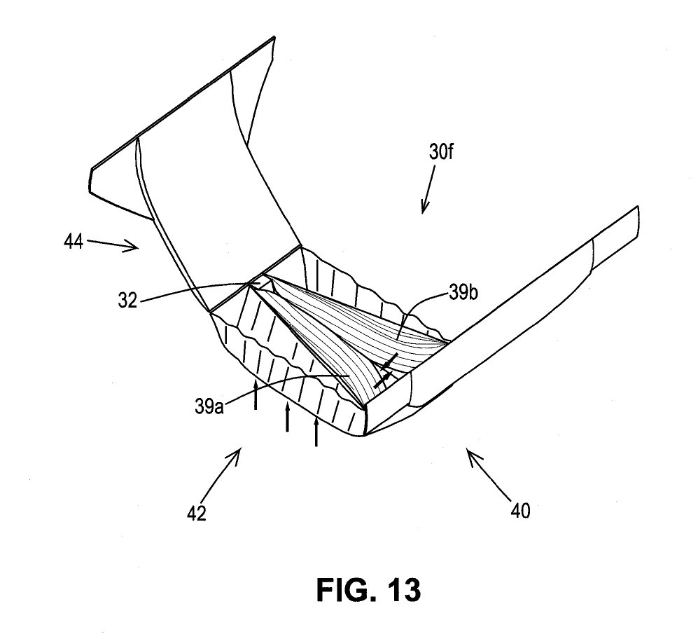

Referring now to FIG. 12, there is shown diaper 30f with air-filled inner cuffs 39a, 39b. When the user sits down, his/her weight applies pressure on the cuffs so that the cuffs expand to the sides until the cuffs contact each other and thus close the pocket area 34 shut (as seen in FIG. 13). This way, the waste contained in pocket 34 does not contact the skin. When the user stands up again, the cuffs return to their original size and position, and do no longer close the pocket area 34. The cuffs are expanded every time the user sits down.

The air-filled cuffs 39a-b are comfortable and soft to the touch.

Referring now to FIG. 13, there is shown diaper 30f as described in FIG. 12, showing air-filled inner cuffs 39a, 39b expanded by applied weight pressure of user.

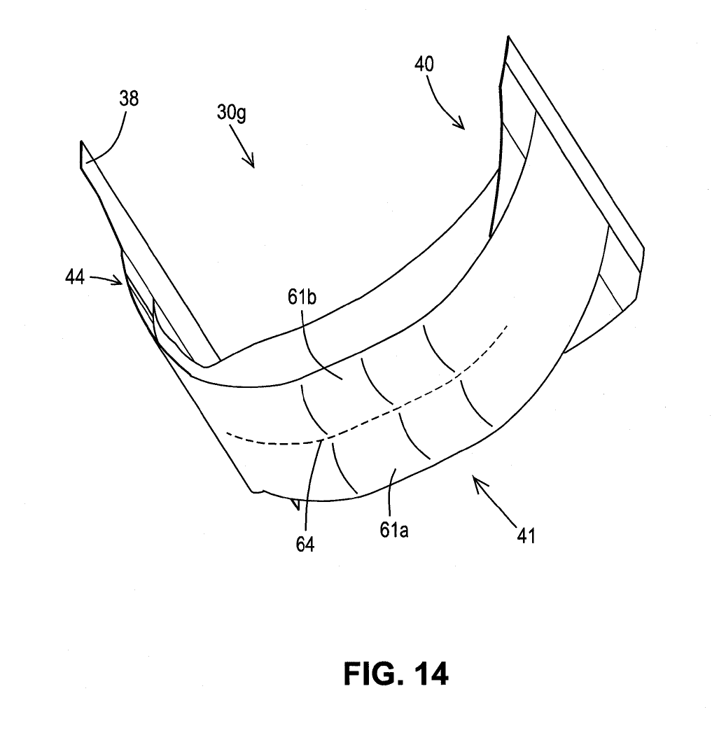

Referring now to FIG. 14, there is shown a bottom view of the inventive diaper 30, showing the external side 41 of crotch area 42, illustrating a stitch 64 extended along the length of diaper 30 which slightly lifts the middle of the diaper and as a result creating two compartments 61a, 61b, on either side of stitch 64, for the purpose of receiving the solid waste into the sides of diaper 30, so as not to have the users skin contact the waste.

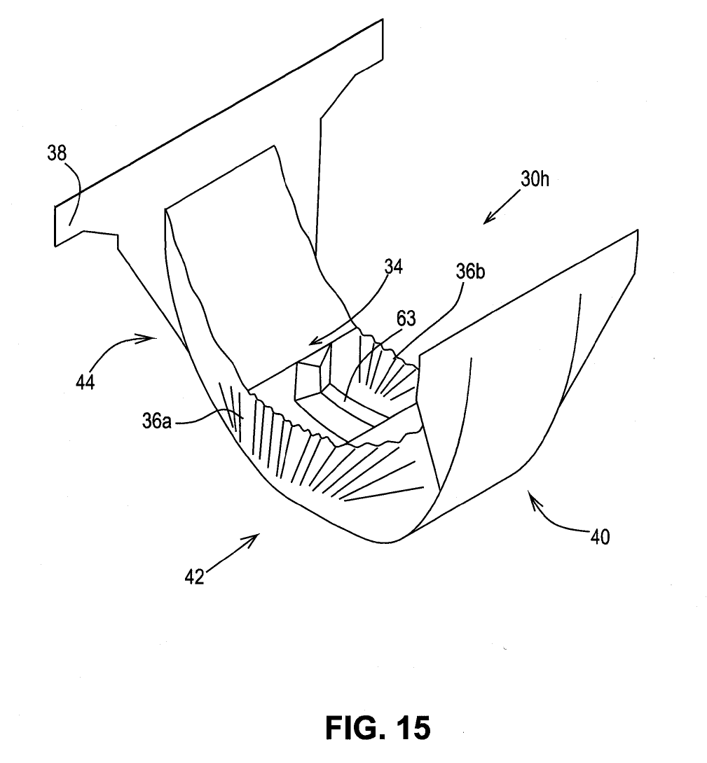

Referring now to FIG. 15, there is shown a perspective view of diaper 30h, illustrating a prism-shaped semi-rigid element 63, positioned similarly to element 32 of FIG. 1, for the purpose of directing solid waste as it rolls along the side of said prism and into the sides of crotch area 42 and into pocket 34, so that the waste does not contact the user's skin.

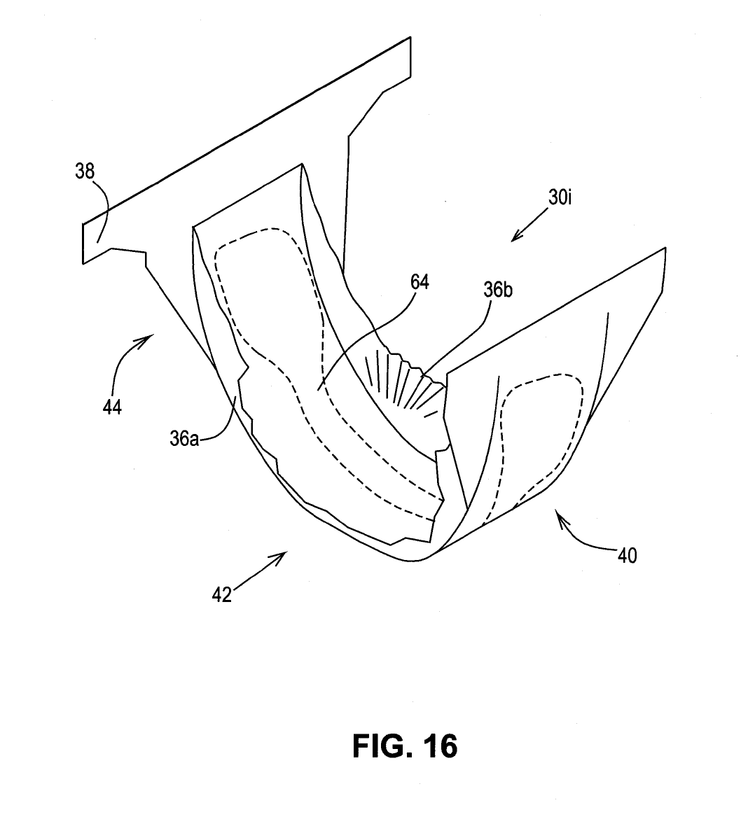

Referring now to FIG. 16, there is shown diaper 30i defining an area with added absorbency 65, though not necessarily visible to the naked eye, starting at front area 40 continuing at crotch area 42 and ending at rear area 44. The area of added absorbency 65 is fabricated of a mixture of SAP (Superabsorbent polymers) and a lignocellulosic fibrous material prepared by chemically or mechanically separating cellulose fibers from wood, fiber crops or paper waste, named Pulp. This mixture is typically used in women's hygienic pads.

In addition, this mixture conducts liquid so that the urine is dispersed evenly across diaper 30i.

Area 65 may be fabricated of any other suitable material having absorbency characteristics.

This particular embodiment of diaper 30i may or may not include a semi-rigid element.

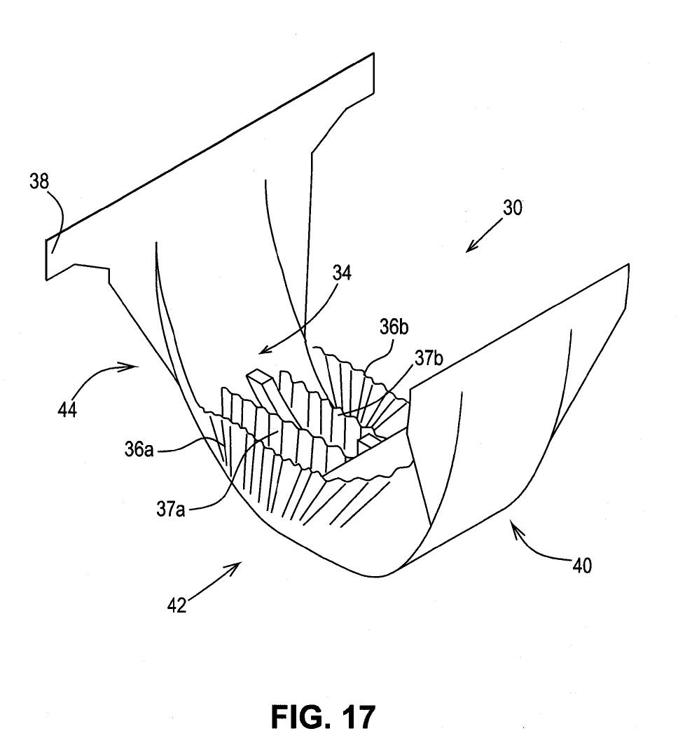

Referring now to FIG. 17, there is shown diaper 30 as described in FIG. 2, with inner cuffs 37a-b positioned from the middle of diaper 30 to the rear end 44 in a parallel fashion, and the edges of inner cuffs 37a-b are slightly slanted inwards, so that when the user sits down, the cuffs 37a-b will be pressed downwards and will not allow the waste to contact the body. Inner cuffs 37a-b are in close contact with the body, and are made sufficiently long so that the waste is securely contained inside pocket 34. Inner cuffs 37a-b can be in close contact with the body, or not in close contact.

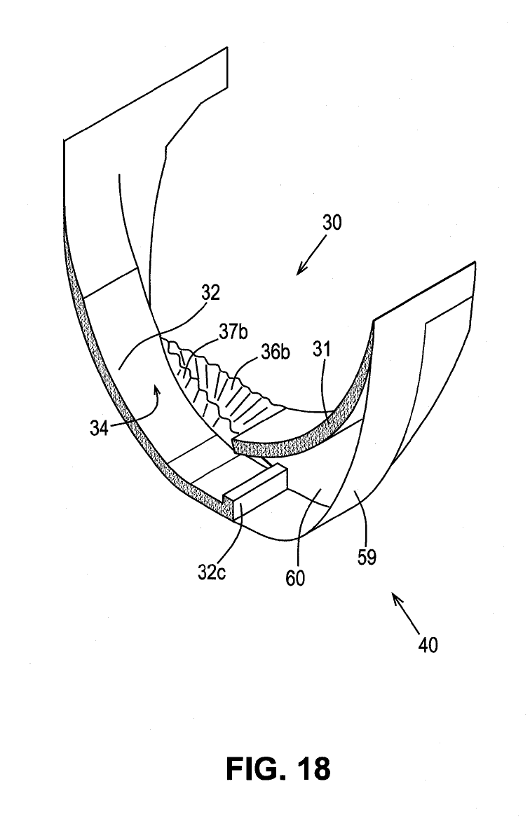

Referring now to FIG. 18, there is shown a cross-section of diaper 30 as described in FIG. 3, illustrating semi-rigid element 32c positioned width-wise to pocket 34, for the purpose of defining and maintaining the structure of diaper 30. Element 32c may be constructed as a three-sectional element as described for semi-rigid element 32 in FIG. 1, or as shown in FIG. 18 as a single-section element.

Having described the invention with regard to certain specific embodiments thereof, it is to be understood that the description is not meant as a limitation, since further modifications may now suggest themselves to those skilled in the art, and it is intended to cover such modifications as fall within the scope of the appended claims.

What is claimed is:

1. An anti-irritant disposable diaper comprising:
a diaper element having an interior, a top area, a front area, a crotch area, a rear area, leg cuffs and closing means;
a semi-rigid element extending from a top front edge of said front area of said diaper element, along said crotch area, to a top rear edge of said rear area, said semi-rigid element defining and maintaining a gap extending along a user contour; and
a solid waste containment means integrally formed within said crotch area and disposed below said gap for receiving excreted solid waste, thus substantially preventing contact of a user's skin with solid waste.

2. The diaper of claim 1, wherein said semi-rigid element comprises a sponge or any other suitable material for maintaining the shape of said containment means.

3. The diaper of claim 1, wherein said semi-rigid element comprises a longitudinal element having joints formed therein along its length so as to define three sections, each with a different orientation: a front section which is substantially vertical, a middle section which is substantially horizontal and a rear section which is substantially vertical, and wherein said semi-rigid element's purpose is maintaining said shape of said containment means.

4. The diaper of claim 1, wherein said front area closely contacts the user's body.

5. The diaper of claim 1, wherein said front area is thick for added absorbency.

6. The diaper of claim 1, wherein said rear area is thick for added absorbency.

7. The diaper of claim 2, wherein said semi-rigid element is wrapped with the absorbing material typically constructing a diaper.

8. The diaper of claim 1, wherein said semi-rigid element is inserted inside said diaper so it is not visible.

9. The diaper of claim 1, wherein said semi-rigid element is inserted all the way through said diaper so it is external to said diaper.

10. The diaper of claim 1, wherein said waste containment means comprises a partition situated above said containment means, said partition having an X-shaped opening to enable passage of the waste.

11. The diaper of claim 1, wherein said waste containment means comprises a semi-rigid element shaped in such a way that while the user is sitting, said front area is folded over said containment means so that it is sealed and its contents cannot contact the user's skin.

12. The diaper of claim 1, wherein said diaper further comprises at least one cuff placed internally and proximate to said leg cuffs, and over said crotch area, wherein said inner cuff comprises a sheet of material for substantially covering solid waste received in said containment means, so that the waste is substantially prevented from contacting the user's skin.

13. The diaper of claim 12, comprising a plurality of inner cuffs positioned diagonally to each other at various angles, or parallel to each other, at varying proximities to each other and at various heights.

14. The diaper of claim 1, wherein said containment means comprises a moveable top cover, and a pullable string connected to said cover to close it over said containment means wherein the user pulls said string for removing said cover and revealing said containment means.

15. The diaper of claim 1, wherein said diaper comprises a scented sticker placed externally on said diaper.

16. The diaper of claim 13, wherein said inner cuffs are filled with air, so that when the user sits the pressure on said cuffs will cause them to expand to a point where said crotch-area is sealed.

17. The diaper of claim 1, wherein said diaper further comprises an absorbing material extending from a top front edge of said front area of said diaper element, along said crotch area, to a top rear edge of said rear area, for enhanced absorbency and dispersing liquid waste evenly over said diaper.

18. The diaper of claim 17, wherein said absorbing material comprises a mixture of Superabsorbent polymers (SAP) and a Lignocellulosic fibrous material (Pulp).

19. The diaper of claim 17, wherein said absorbing material comprises any biocompatible absorbing material suited for diapers.

20. The diaper of claim 1, wherein said crotch area is narrow, for added comfort of the user.

21. The diaper of claim 1, wherein said diaper comprises a bottom side of said crotch area, wherein said bottom side comprises a stitch extended along the length of said bottom side, wherein said stitch lifts the middle of said crotch area resulting in a compartment for receiving solid waste, on either side of said stitch, for substantially preventing contact of a user's skin with solid waste.

22. The diaper of claim 3, wherein said semi-rigid element is shaped as a prism and the solid waste is directed sideways as it rolls along the side of said prism and into said containment means.

23. The diaper of claim 1, wherein said semi-rigid element is shaped as a longitudinal section, placed on said rear area and on said front area, and wherein said longitudinal sections are spaced apart from each other.

24. The diaper of claim 12, wherein said inner cuffs comprise a pullable string, so that when said string is pulled, said inner cuffs move towards each other and are joined and so said containment means are covered.

25. The diaper of claim 1, wherein said containment means comprises a moveable top cover, and a pullable string connected to said cover to close it over said containment means wherein the user pulls said string for removing said cover and revealing said containment means.

26. The diaper of claim 2, wherein said semi-rigid element is positioned width-wise to said containment means, for the purpose of defining and maintaining said structure of said diaper.

27. An anti-irritant disposable diaper comprising:
a diaper element having an interior, a top area, a front area, a crotch area, a rear area, leg cuffs and closing means;
a gap extending along a user contour and above said crotch area;
a solid waste containment means integrally formed within said crotch area and disposed below said gap for receiving excreted solid waste, thus substantially preventing contact of a user's skin with solid waste; and
at least one cuff placed internally and proximate to said leg cuffs, and over said crotch area, wherein said inner cuff comprises a sheet of material for substantially covering solid waste received in said containment means, so that the solid waste is substantially prevented from contacting the user's skin.

28. A sanitary device for providing protection against irritation to the skin, said device comprising:
a disposable diaper, comprising:
a diaper element having an interior, a top area, a front area, a crotch area, a rear area, leg cuffs and closing means;
a semi-rigid element extending from a top front edge of said front area of said diaper element, along said crotch area, to a top rear edge of said rear area, said semi-rigid element defining and maintaining a gap extending along a user contour; and
a solid waste containment means integrally formed within said crotch area and disposed below said gap for receiving excreted waste, thus substantially preventing contact of a user's skin with solid waste.
